(19) United States
(12) Patent Application Publication
(10) Pub. No.: US 2009/0092639 A1
(43) Pub. Date: Apr. 9, 2009

(54) IMMUNOGENIC COMPOSITION

(76) Inventors: Mark E. Cook, Madison, WI (US); David L. Trott, Madison, WI (US); Mingder Yang, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 12/337,123

(22) Filed: Dec. 17, 2008

Related U.S. Application Data
(62) Division of application No. 11/586,136, filed on Oct. 25, 2006.

(51) Int. Cl. A61K 39/00; A61K 35/54

(52) U.S. Cl. 424/243.1; 424/234.1; 424/247.1; 424/581

(57) ABSTRACT

Methods and compositions for increasing antibody titer in egg yolks and for using the antibodies are disclosed.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

The invention relates generally to compositions and methods for producing antibodies in avian animals, and more particularly to compositions and methods for achieving higher specific antibody titers in eggs than can be achieved using current compositions and methods.

Avian antibodies offer advantages over mammalian antibodies. First, avian antibodies are easily obtained and isolated from eggs. Second, avian antibodies are cost-effective to produce. Third, avian antibodies are biochemically advantageous over mammalian antibodies because of phylogenetic differences between avian and mammalian species. The skilled person is generally familiar with methods for obtaining antibodies, including human monoclonal antibodies, from avian egg yolks. See Bar-Joseph M & Malkinson M, “Hen egg yolk as a source of antiviral antibodies in the enzyme-linked immunosorbent assay (ELISA): a comparison of two plant viruses,” J. Virol. Methods 1: 179-183 (1980); Gassmann M, et al., “Efficient production of chicken egg yolk antibodies against a conserved mammalian protein,” FASEB J. 4:2528-32 (1990); and Zhu L, et al., “Production of human monoclonal antibody in eggs of chimeric chickens,” Nature Biotechnology 23:1159-1169 (2005), each of which is incorporated herein by reference as if set forth in its entirety.

Typically, an avian animal is immunized with an immunogenic composition containing an antigen against which antibodies are raised, an adjuvant that stimulates the animal's immune response and a suitable immunologically inert carrier. As needed, one or more boosts of a similar composition are administered several days or weeks after the initial immunization. A typical adjuvant in the immunogenic composition is Freund's Complete Adjuvant (FCA). Boosts typically contain Freund's Incomplete Adjuvant (FIA) in place of the FCA. Antibodies are generated in serum and are transported to eggs. The eggs are harvested and egg yolks are prepared and stored (e.g., as a powder) for subsequent use. Methods for preparing egg yolk powder containing useful antibodies are known and need not be detailed. As needed, the egg yolk antibodies can be separated from the egg yolks to a level of purity suited for the use to which the antibodies will be put. Typical uses include use as research tools, diagnostic agents and therapeutic agents, including as agents for transmitting passive immunity.

Higher antibody titer in the egg yolks from immunized avian animals translates directly into increased production efficiency, for example, by decreasing the number of times that a human must immunize the animals, harvest the eggs and prepare the yolks and antibodies. Additionally, at each doubling of specific antibody titer in an egg, the cost of producing the antibody product is reduced by 50%. Accordingly, immunogenic compositions and methods for producing egg yolks having appreciably higher specific antibody titer than is currently available continue to be sought in the art.

Certain bacterial components are hypothesized to modulate immune response in poultry when included in the administered immunogenic composition. Parmentier H, et al., “Differential effects of lipopolysaccharide and lipoteichoic acid on the primary antibody response to keyhole limpet hemocyanin of chickens selected for high or low antibody responses to sheep red blood cells,” Poult. Sci. 83:1133-1139 (2004). Parmentier et al. intravenously injected lipopolysaccharide (LPS) or lipoteichoic acid (LTA) into two strains of male chickens twenty-four hours before a subcutaneous injection of keyhole limpet hemocyanin (KLH). Although Parmentier et al. concluded that LPS attenuated, and that LTA enhanced, antibody titer in serum, these results should be interpreted with caution, as KLH is a known immunostimulant. See McFadden D, et al., “Keyhole limpet hemocyanin, a novel immune stimulant with promising anticancer activity in Barrett's esophageal adenocarcinoma,” Am. J. Surg. 186:552-555 (2003). In fact, Parmentier et al. showed that KLH alone increased anti-LPS and anti-LTA antibody titer in chicken serum, even without exposing the chicken to LPS or LTA. Also, Parmentier et al. used two chicken strains that were bred for high- or low-antibody response. More importantly, Parmentier et al.'s method of increasing antibody titer in poultry is cost-prohibitive on a large-scale because it requires multiple injections and multiple handlings of poultry within twenty-four hours of antigen presentation.

Moreillon P & Majcherczyk P, “Proinflammatory activity of cell-wall constituents from gram-positive bacteria,” Scand. J. Infect. Dis. 35:632-641 (2003), discussed structural aspects of gram-positive bacterial wall components involved in inflammatory response and reported on the amino acid composition of peptidoglycan in various gram-positive bacteria, noting in Table III and accompanying text a statistically significant relationship between a bacterium's natural habitat and its diamino acid structure at position 3 of the peptidoglycan.

A need for compositions and methods to increase egg yolk antibody titer persists.

### SUMMARY

In one aspect, the present invention is summarized in that an immunogenic composition contains an antigen against which antigen-specific antibodies are raised, an adjuvant that stimulates the animal's immune response, at least one gram-positive bacterium and a suitable immunologically-inert, pharmaceutically-acceptable carrier.

In another aspect, the present invention is further summarized in that a method for producing antibodies in eggs of an avian animal includes the step of immunizing the animal with the immunogenic composition. Surprisingly, the immunogenic composition increases antigen-specific egg yolk antibody titer to a titer higher than that in egg yolks obtained from an animal immunized with an immunogenic composition that lacks the gram-positive bacterium, when the composition is administered in a method to produce antigen-specific egg yolk antibodies. In such a method, the amount of the antigen is an immunogenic amount and the amount of the gram-positive bacterium in the composition is an amount effective to increase the antigen-specific titer to a level higher than that obtained using an immunogenic composition lacking the bacterium. An effect of the gram-positive bacterium in the composition is observed between twenty-one and forty-two days after initial immunization. Immunization can be by intramuscular injection or other suitable delivery approach.

In some embodiments, the gram-positive bacterium is a Clostridium bacterium, such as C. perfringens, or a Staphylococcus bacterium, such as S. aureus.

In some embodiments, the avian animal is a chicken, duck, emu, goose, ostrich, pheasant, quail or turkey.

Some embodiments of the method include a second step of administering to the animal at least one booster immunization with the immunogenic composition after the initial administration, in accord with conventional booster protocols. In some cases, the booster is administered at least about seven days or at least about fourteen or at least about twenty-eight days after the initial immunization.

In some immunization steps, especially in the initial immunization, the adjuvant in the immunogenic composition is Freund's Complete Adjuvant. Alternatively, the adjuvant can be Freund's Incomplete Adjuvant.

Other features, aspects and advantages of the present invention will become better understood from the description that follows. The description of preferred embodiments is not intended to limit the invention to cover all modifications, equivalents and alternatives. Reference should therefore be made to the claims herein for interpreting the scope of the invention.

### BRIEF DESCRIPTION OF THE DRAWINGS

Not applicable.

### DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are now described.

As used herein, an immunogenic amount is defined as a concentration that causes an animal to produce antibodies against an antigen.

As used herein, an effective amount is defined as a concentration that causes an antibody titer in an egg from an avian animal immunized in accord with the methods disclosed herein to be higher than that in an egg from an avian animal immunized with an immunogenic composition lacking a gram-positive bacterium. For example, a level at least twice as high can be achieved when an effective amount of a gram-positive bacterium is included in the composition.

As used herein, a gram-positive bacteria is defined as those bacteria that retain a crystal violet dye during the gram stain process. Specifically excluded from this definition is Mycobacterium tuberculosis.

As used herein, a pharmaceutically acceptable carrier is a standard non-immunogenic pharmaceutical carrier. Examples of suitable carriers are well known in the art and may include, but are not limited to, any of the standard pharmaceutical carriers such as citrate-buffered saline solutions, phosphate-buffered saline solutions, phosphate-buffered saline containing Polysorb 80, water, emulsions such as oil/water emulsion, and various types of wetting agents.

As used herein, an arbitrary unit (AU) means the highest dilution of egg yolk containing antibody that will give an absorbance twice that of egg yolk from egg of hens not inoculated with the immunogen or antigen, which in this instance was PLA₂.

Antibody titer can be determined in egg yolks using the following approach. For purposes of the invention, the important measure is a relative antibody titer rather than an absolute titer. Antibodies can be extracted by a 1:10 dilution of yolk in acidified PBS (pH 5.0) for twelve hours and assayed by enzyme-linked immunosorbent assay (ELISA). Optical density or absorbance can be measured at 450 nm using an horseradish peroxidase enzyme reaction to detect antigen-specific anti-PLA₂ antibody. A “positive reaction” standard on each plate can be defined as twice the optical density or absorbance at 450 nm of an egg yolk diluted 1:2000 from a hen not immunized against the antigen. A control value was established by measuring the amount of anti-PLA₂ antibody in eggs from hens that were not immunized.

Without intending to be limited to any particular theory of the invention, it is thought that the presence of gram-positive bacteria provides in the immunogenic composition a structural mixture of cell wall components that further enhance the immunogenicity of the composition, and that the cell wall components of bacterial components in conventional adjuvants (for example, the acid fast M. tuberculosis of FCA) are deficient in some structural regard relating to immunogenicity relative to those of gram-positive bacteria. Again without being limited as to theory, it is thought that a component of gram-positive peptidoglycan, perhaps a disaccharide-pentapeptide subunit or a muramyl dipeptide, plays an important role in establishing the immunogenicity contributed by the gram-positive bacterium.

To ensure sufficient antibody production, at least 50 ug of a gram-positive bacterium is desired in the antigenic preparations described below. However, it is contemplated that up to 10 mg of the gram-positive bacteria is acceptable. Combinations of two or more gram-positive bacteria can be used.

The invention will be more fully understood upon consideration of the following non-limiting Examples.

### EXAMPLES

### Example 1

### Increased Antibody Titer in Chicken Eggs Resulting from Administration Of an Effective Concentration of Gram-Positive Bacteria at Time of Immunization

Single Comb White Leghorn hens were randomly assigned to treatment groups. The hens had free access to standard breeder's mash and water.

A group of hens (n=8) received an intramuscular (i.m.) injection of 3 mg PLA₂/ml of an emulsion comprising one part FCA and one part C. perfringens type C or S. aureus (Novartis Animal Health, Inc.; Greensboro, N.C.). At days fourteen and twenty-eight, the hens received a booster emulsion having an equal volume of FIA and 3 mg PLA₂ in an aqueous solution. Antibody titer on day 28 was increased 1.25 fold in C. perfringens-treated hen and 1.58 fold in S. aureus-treated over control in eggs, respectively.

A group of hens (n=8) received an i.m. injection of 3 mg PLA₂/ml of an emulsion comprising one part FCA and one part S. aureus. At day seven, the hens received a booster of FIA with PLA₂. Antibody titer on day 28 was 1.7 fold higher than control.

A group of hens received an i.m. injection of 3 mg PLA₂/ml of an emulsion comprising one part FCA and one part E. coli. At day seven, the hens received a booster of FIA with PLA₂. Antibody titer on day 28 was reduced an average of 42% compared to control. This indicates that gram-negative bacteria do not increase relative titer, as is shown for gram-positive bacteria.

A group of hens (n=8) received an i.m. injection of 3 mg PLA₂/ml of an emulsion comprising one part FCA and one part S. aureus. At days fourteen and twenty-eight, the hens received a booster emulsion having an equal volume of FIA and 3 mg PLA₂ in an aqueous solution. Antibody titer on day 42 was 1.6 fold higher in treated eggs than in eggs receiving the same vaccine without S. aureus. When hens received the above described control and S. aureus vaccines, but boosted only on day 7 (instead of days 14 and 28) using the FIA with PLA₂, eggs from hens getting the S. aureus in the first injection was 2.4 higher than those eggs from hens not receiving PLA₂ with S. aureus during the first vaccination.

In a separate trial, hens received an i.m injection having S. aureus as described above. A booster was given day seven as described above. An increase in antibody titer over control (i.e., FCA alone) began at day 28, and was maintained at days 35, 42, 49, 56, 63, and 70. The average antibody titer increase in S. aureus hens was 1.7 fold for all days beginning with day 28.

### Example 2

### Feed Supplement

Feed supplements are one way to enhance animal productivity and animal health. These supplements are administered to animals for preventing and treating infectious disease, for promoting growth, for improving feed conversion and for increasing the yield of useful products, such as meat, milk and eggs.

One way to prevent or treat infectious disease is by using antibiotics as a feed supplement. Unfortunately, antibiotics present several disadvantages. For one, over-use of antibiotics leads to drug-resistant pathogens in animals. Additionally, antibiotics can accumulate in animal products. When humans consume the animal products containing antibiotics, the antibiotics can likewise lead to drug-resistant pathogens in humans. Finally, antibiotics are expensive to develop and manufacture, as significant amounts of time and money are invested to develop new antibiotics. The expense is multiplied if new antibiotics must be developed to overcome drug-resistant pathogens.

Because of the disadvantages associated with using antibiotics as feed supplements, antibodies are preferred as feed supplements for preventing and treating disease. Antibodies, which are a natural component of humoral immunity, do not lead to resistance in pathogens. Additionally, antibodies are less expensive to develop and manufacture than antibiotics for use in feed supplements.

To manufacture a feed supplement, antibodies are produced according to the method described in Example 1. The antigen used to produce the antibodies is, e.g., an epitope of a pathogen, which can be, without limitation, a virus, a bacteria, a fungus or a protozoan. To recover a usable antibody producer from an egg, albumin and yolk are dried to form a shelf-stable egg powder that contains antibodies that may be used as a feed supplement to prevent an animal from becoming infected.

### Example 3

### Vaccine for Passive Immunity

Vaccines are used to produce the same immunological result—production of antibodies—in a subject without making the subject suffer through a disease.

There are two broad categories of vaccines, active and passive. An active vaccine stimulates a subject's immune system to produce antibodies or cellular immune responses or both to protect against or eliminate a disease. Conversely, a passive vaccine is a preparation of antibodies to neutralize a pathogen and is administered before or around the time of known or potential exposure. Although active vaccines are generally preferred, passive vaccines may be required in specific instances, especially if no active vaccine is available or if the subject is immuno-compromised.

To manufacture a passive vaccine, antibodies are produced according to the method described in Example 1. The antigen used to produce the antibodies is, e.g., an epitope of a pathogen, which can be, without limitation, a virus, a bacteria, a fungus, a protozoan or a cancer. Methods to recover antibodies in substantially pure form suitable for a vaccine are known to those of skill in the art of vaccine production. The substantially pure antibodies are then mixed with a suitable adjuvant and administered to a subject.

### Example 4

### Diagnostic Kit

To manufacture a diagnostic kit, antibodies are produced according to the method described in Example 1. The antigen used to produce the antibodies is directed to an epitope of a desired target for the assay. Methods to recover antibodies in substantially pure form suitable as a reagent in a diagnostic kit are known to those of skill in the art of vaccine production.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

1. A method for increasing antigen-specific antibody titer in an egg yolk of an avian animal, the method comprising the step of:
administering an immunogenic composition to the animal, the composition comprising an immunologically-inert, pharmaceutically-acceptable carrier, an immunogenic amount of an antigen against which antigen-specific antibodies are raised, an adjuvant that stimulates the animal's immune response, an amount of at least one gram-positive bacterium effective to increase the antigen-specific titer relative to that achieved in an animal administered an immunogenic composition lacking the gram-positive bacterium.

2. A method as recited in claim 1, wherein the avian animal is selected from the group consisting of a chicken, a duck, an emu, a goose, an ostrich, a pheasant, a quail and a turkey.

3. A method as recited in claim 1 wherein the avian animal is a chicken.

4. A method as recited in claim 1, wherein the gram-positive bacterium is selected from the group consisting of a Clostridium bacterium and a Staphylococcus bacterium.

5. A method as recited in claim 1, wherein the gram-positive bacterium is selected from the group consisting of Clostridium perfringens and Staphylococcus aureus.

6. A method as recited in claim 1, wherein the gram-positive bacteria is Staphylococcus aureus.

7. A method as recited in claim 1, wherein the gram-positive bacteria is Clostridium perfringens.

8. A method as recited in claim 1, further comprising at least one booster administration.

9. A method as recited in claim 8, where the at least one booster administration is delivered at about day seven after the immunogenic composition is administered.

10. A method are recited in claim 8, wherein a first booster administration is delivered at about day fourteen and a second booster administration is delivered at about day twenty-eight after the immunogenic composition is administered.

11. A method as recited in claim 1, wherein the adjuvant is Freund's complete adjuvant.

12.-20. (canceled)

21. A method for producing an avian egg yolk containing antibodies specific for an immunizing antigen, the method comprising the step of:
administering to the animal an immunogenic composition that comprises, in an immunologically-inert, pharmaceutically-acceptable carrier, an immunogenic amount of the immunizing antigen, an adjuvant that stimulates the animal's immune response, and at least one gram-positive bacterium distinct from the immunizing antigen in an amount effective to yield the antibodies specific for the immunizing antigen at a titer higher in the egg yolk than in an egg yolk of an avian animal administered the immunizing antigen in an immunogenic composition that comprises, in the immunologically-inert, pharmaceutically-acceptable carrier, the immunogenic amount of the immunizing antigen and Freund's complete adjuvant but which lacks the at least one gram-positive bacterium.

22. The method of claim 21, wherein the titer is at least 1.25 fold higher in the yolk of the avian animal administered the at least one gram-positive bacterium.

23. A method for making an egg yolk powder containing antibodies specific for an immunizing antigen, the method comprising the steps of:
administering to the animal an immunogenic composition that comprises, in an immunologically-inert, pharmaceutically-acceptable carrier, an immunogenic amount of the immunizing antigen, an adjuvant that stimulates the animal's immune response, and at least one gram-positive bacterium distinct from the immunizing antigen in an amount effective to yield the antibodies specific for the immunizing antigen; and
drying the yolk of the avian animal administered the composition that comprises the at least one gram-positive bacterium to produce the egg yolk powder,
the antibodies specific for the immunizing antigen being present at a titer higher in the egg yolk than in an egg yolk of an avian animal administered the immunizing antigen in an immunogenic composition that comprises, in the immunologically-inert, pharmaceutically-acceptable carrier, the immunogenic amount of the immunizing antigen and Freund's complete adjuvant but which lacks the at least one gram-positive bacterium.

24. An egg yolk powder produced according to a method comprising the steps of:
administering to an avian animal an immunogenic composition that comprises, in an immunologically-inert, pharmaceutically-acceptable carrier, an immunogenic amount of the immunizing antigen, an adjuvant that stimulates the animal's immune response, and at least one gram-positive bacterium distinct from the immunizing antigen in an amount effective to yield the antibodies specific for the immunizing antigen; and
drying the yolk of the avian animal administered the composition that comprises the at least one gram-positive bacterium to produce the egg yolk powder,
the antibodies specific for the immunizing antigen being present at a titer higher in the yolk than in an egg yolk of an avian animal administered the immunizing antigen in an immunogenic composition that comprises, in the immunologically-inert, pharmaceutically-acceptable carrier, the immunogenic amount of the immunizing antigen and Freund's complete adjuvant but which lacks the at least one gram-positive bacterium.

25. The powder of claim 24, wherein the titer is at least 1.25 fold higher in the yolk of the avian animal administered the at least one gram-positive bacterium.
